(19) United States
(12) Patent Application Publication
(10) Pub. No.: US 2012/0150092 A1
(43) Pub. Date: Jun. 14, 2012

(54) GUIDED PERCUTANEOUS BYPASS

(75) Inventors: Todd N McAllister, San Anselmo, CA (US); Sergio A Garrido, Buenos Aires (AR); Nicolas L'Heureux, Corte Madera, CA (US)

(73) Assignee: CYTOGRAFT TISSUE ENGINEERING, INC., Novato, CA (US)

(21) Appl. No.: 13/256,409

(22) PCT Filed: Mar. 17, 2010
(86) PCT No.: PCT/US10/27717

§ 371 Date: Feb. 27, 2012

Related U.S. Application Data
(60) Provisional application No. 61/202,602, filed on Mar. 17, 2009.

(51) Int. Cl. A61M 25/16; A61M 25/09

(52) U.S. Cl. 604/8

(57) ABSTRACT

The invention includes methods and apparatus to deploy a blood vessel conduit via a catheter-based, percutaneous approach. In particular, a prosthetic blood conduit can be introduced around or through an arterial obstruction without requiring open bypass surgery. The technology includes coupling devices for docking the tips of two catheters, one situated inside a blood vessel, the other situated outside the blood vessel wall.

### CLAIM OF PRIORITY

This application claims the benefit of priority under 35 U.S.C. § 119(e) to U.S. provisional application Ser. No. 61/202,602, filed Mar. 17, 2009, which is incorporated herein by reference in its entirety.

### TECHNICAL FIELD

The technology described herein generally relates to the field of endovascular repair of cardiovascular disease, and more particularly addresses a device and methods to deliver and deploy a blood vessel conduit via a catheter-based, percutaneous approach. That is, the technology includes a device that can place a prosthetic blood conduit around or through an obstruction without requiring open bypass surgery.

### BACKGROUND

Coronary and peripheral vascular disease cumulatively affect more than 10% of the U.S. population. The primary manifestation of vascular disease is a narrowing of arteries due to one or both of calcified atherosclerotic lesions or hyper proliferation of vascular smooth muscle cells. In both cases, the narrowing restricts blood flow to the distal tissues which can lead to myocardial infarction, lower limb ischemia, etc. If the arterial narrowing reaches a critical level and there are clinically relevant manifestations, blood flow can usually be restored by surgically placing a new vessel which re-routes blood flow around the blockage (a bypass). In many cases, blood flow can alternatively be restored percutaneously using less invasive catheter-based technologies to open, ablate, or remove the stenotic lesions.

There are a variety of potential advantages to the less invasive percutaneous repair technique, in particular shorter hospital stays, and a trend toward lower mortality rate as the procedures are more widely deployed, both of which have driven rapid clinical adoption. Today, more than 1 million percutaneous procedures are performed in the U.S. each year.

In many cases, however, percutaneous disruption of the arterial narrowing is not recommended, and an open, surgical bypass must be performed. While an open bypass procedure is significantly more invasive, most studies show a clear long-term increase in efficacy relative to catheter based treatments. Today more than 300,000 coronary bypasses, and more than 100,000 peripheral bypasses, are performed annually in the U.S.

Over the last several years, the line between classic open surgical bypass and endovascular approaches has become blurred, as techniques to deliver a prosthetic conduit less invasively have been developed. These less invasive techniques have generally focused on re-lining a diseased vessel with a segment of saphenous vein (or other autologous tissue) delivered via a catheter introduced through the femoral artery. Similarly, ePTFE wrapped stent grafts can be delivered and deployed to re-line the diseased artery percutaneously. Additionally, robotically assisted coronary bypasses have been performed thoracoscopically, i.e., without open surgery but using minimal entry points and operations guided by live imaging technology.

In general, percutaneous re-lining of diseased arteries has been limited to peripheral (non-coronary) applications, and typically utilizes a synthetic conduit that has an expandable stent at either end to anchor the device in the diseased vessel. The device is delivered by deploying the stent at one end, then traversing though the arterial blockage, then deploying a second stent at the other end. In some cases, the stent is a single piece that stretches the length of the graft. These devices typically cross through the occlusive lesion if the lesion is mechanically disrupted, or they can traverse around the lesion by going through the subintimal space (i.e., in between the intima and the adventitia of the vessel).

Previous percutaneous approaches have not been applicable to coronary applications because the coronary arteries are much narrower than the peripheral vessels in question. This creates difficulties in delivery, but more importantly, existing prosthetic materials tend to thrombose (clot) in these smaller diameter applications. Because of these limitations, percutaneous revascularization (bypass) is typically limited to above knee bypass (femoral or iliac arteries) or carotid bypass where both the diameter and the vessel architecture is such that both the proximal and distal ends of the bypass graft can be deployed inside the same native vessel.

In coronary applications, for example, a classic bypass would use a vein graft sewn to the aorta on the proximal end with an end to side anastomosis, and a small diameter target coronary vessel on the distal end also sewn with an end to side anastomosis. Alternatively, an internal mammary artery (IMA) is cut from the subclavian branch and swung back to the coronary vasculature instead. Existing percutaneous strategies have not been attempted to perform a coronary bypass because in these smaller vessels, no synthetic conduit can be used to simply bypass around or through the small coronary arteries (low blood flow in combination with the small diameter leads to thrombosis of the synthetic bypass).

An alternative strategy for percutaneous coronary bypass would be to branch directly off the aorta or one of its large branches (such as the IMA, or the subclavian), but is currently not possible. This type of percutaneous bypass (aorta to distal coronary target) is limited by the fact that bleeding cannot be well controlled once the device is deployed in and perforates the aorta. Moreover, it is extremely difficult to accurately deliver the distal end of the bypass to the proper location in the target vessel, since no provision for real time, intra-operative guidance other than standard two-dimensional angiography is currently available. Finally, this type of bypass would be complicated by the fact that the heart is surrounded by a fluid filled pericardial sac, which creates another navigational obstacle for deployment of the distal end.

Taken together, while the techniques for percutaneous bypass within the luminal space (i.e. intravascular as opposed to perivascular bypass) generally work well in simple peripheral cases where the diseased artery is simply relined, the technologies currently available will not work for small diameter applications in both the peripheral and the coronary circulation, particularly those that require tunneling or navigation through the perivascular space. In particular, the materials that have typically been used are not suitable for these more delicate applications, and there does not currently exist a mechanism for accurately linking up the proximal and distal portions of a percutaneous bypass in connection with the repair of narrow arteries.

The discussion of the background herein is included to explain the context of the technology. This is not to be taken as an admission that any of the material referred to was published, known, or part of the common general knowledge as at the priority date of any of the claims found appended hereto.

Throughout the description and claims of the specification the word “comprise” and variations thereof, such as “comprising” and “comprises”, is not intended to exclude other additives, components, integers or steps.

### SUMMARY

The technology herein includes a method for percutaneously delivering a blood vessel bypass conduit (either biological or synthetic). The method can be used for either coronary or peripheral bypass, and can also be combined with open procedures such that one end of the bypass is connected via laparoscopic or open techniques, while the other end is connected via a percutaneous approach.

The technology described herein includes a method for cardiovascular repair using a guided, steerable system of catheters and guidewires that have provisions for positive guidance and connection.

The technology includes a method for carrying out a percutaneous bypass that allows a bypass conduit to be delivered through the perivascular space. This method allows a surgeon or interventionalist to perforate the vessel proximal to a lesion and navigate through the perivascular space to a specific target point distal to the lesion. The method provides a mechanism to actively assist the surgeon in locating a re-entry point on the vessel at the distal target point. The method is superior to two dimensional guidance via angiography, which is not usually effective for reconstructing the three dimensional architecture of the vasculature.

The method of percutaneous bypass as described herein addresses problems of catheter guidance and device deployment when the blood must be re-routed from one vessel to another or through the perivascular space. Moreover, the technology includes methods to control bleeding after the proximal artery has been opened to allow the device to be routed toward the distal target. The technology allows percutaneous bypass in coronary applications and for complex peripheral bypass where end to side anastomoses would typically be required. The technology also facilitates debranching procedures, where flow is restored to a distal organ using an artery or a proximal target that does not typically feed that organ.

The technology further includes method of performing a percutaneous bypass on a subject, such as a subject in need thereof, the method comprising: docking a first catheter situated inside a damaged vessel to a second catheter situated outside the vessel, at a location downstream of an occlusion in the damaged blood vessel; and inserting a bypass blood vessel over the second catheter.

The technology still further includes a method of performing a percutaneous bypass on a subject, the method comprising: introducing a first catheter into the subject; positioning a first tip of the first catheter at a location distal to an occlusion in a damaged blood vessel; introducing a second catheter into the subject at a location proximal to the occlusion; docking a second tip of the second catheter to the first tip of the first catheter at the location distal to the occlusion; inserting a guidewire down the second catheter so that the guidewire traverses the location distal to the occlusion; withdrawing the second catheter; inserting a length of bypass blood vessel over the guidewire; and joining the bypass blood vessel to the damaged blood vessel at the distal location.

The bypass blood vessel can be produced by a process termed sheet-based tissue engineering. A biological conduit produced by such a process can be used for either open or percutaneous bypass. This material addresses many of the limitations associated with small diameter bypass for both open and percutaneous procedures.

The technology herein further includes an apparatus for coupling two catheters across a blood vessel wall, the apparatus comprising: a first catheter having a first magnet located at its end, a second catheter having a second magnet located at its end; wherein a first surface of the first magnet is complementary to a second surface of the second magnet; wherein the first magnet is strong enough to attract and engage the second magnet when separated from the second magnet by the blood vessel wall, and wherein the second magnet encloses a hole through which a guidewire travels; and wherein the first magnet comprises a chute that accepts the guidewire and deflects the guidewire downstream into the blood vessel.

### BRIEF DESCRIPTION OF THE DRAWINGS

FIG. 1 shows a first of a sequence of depictions of two catheters docking

FIG. 2 shows a second of a sequence of depictions of two catheters docking

FIG. 3 shows a third of a sequence of depictions of two catheters docking

FIG. 4 shows a view of an artery and two catheters docking to one another via magnets.

FIG. 5 shows a cutaway view of a patient's chest showing a bypass vessel in place.

FIG. 6 shows a flow-chart of a process as described herein;

Like reference symbols in the various drawings indicate like elements.

### DETAILED DESCRIPTION

The instant technology is directed to devices and methods to deliver and deploy a blood vessel conduit via a catheter-based, percutaneous approach. That is, the technology includes a device that can place a prosthetic blood conduit around or through an obstruction without requiring open bypass surgery. The technology has particular application to endovascular repair of cardiovascular disease.

### Procedure for Percutaneous Bypass

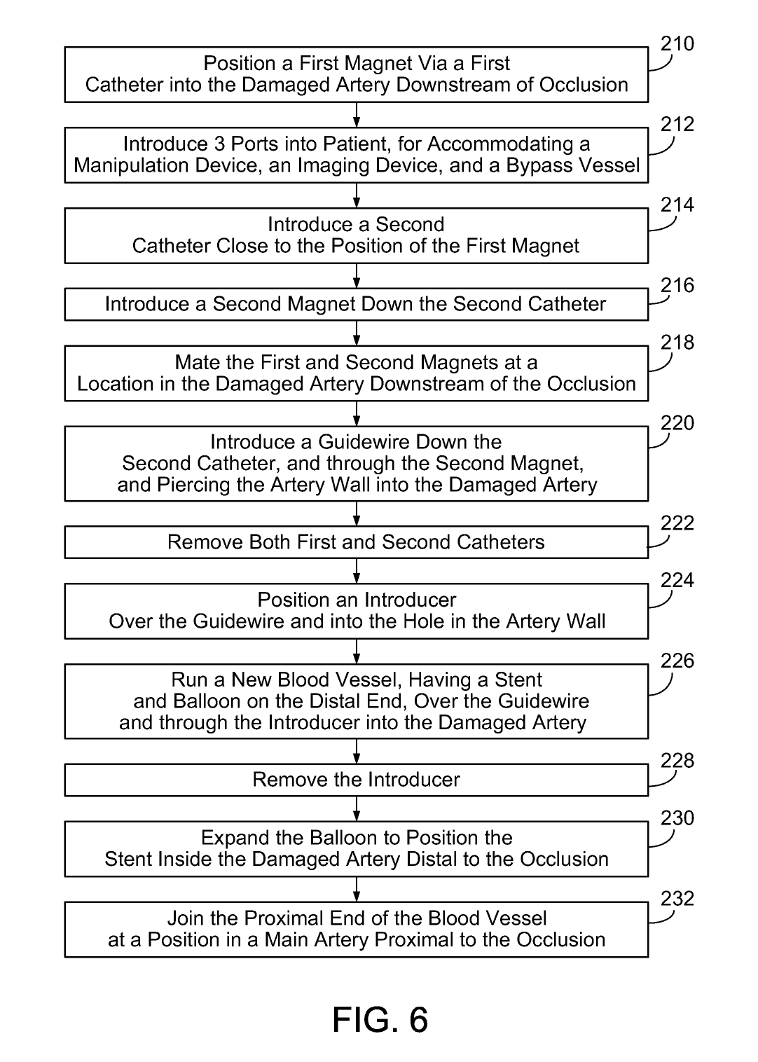

In overview, a process for deploying a blood vessel using a percutaneous approach is described in FIG. 6. It would be understood that the technology is not limited to the precise steps shown in FIG. 6 and that variations thereof, including conflation of certain steps into fewer steps, addition of further steps, or omission of certain steps, remains consistent with the invention.

An artery, such as a peripheral artery, or a coronary artery having an occlusion, and the location of the occlusion, are identified, such as by various diagnostic and/or imaging techniques known in the art.

At 210, a first magnet is positioned via a first catheter into the damaged artery downstream of the occlusion. Typically, the first magnet is situated at the tip of the first catheter. The catheter is inserted into the damaged artery using techniques commonly practiced in surgery. For example, in the case of a coronary artery, the first catheter can be inserted in the wrist, and caused to follow the radial artery, into the aortic artery, followed by the subclavian artery, and then into heart.

The first catheter can be routed around or through the occlusion in the damaged vessel to a position ideal for bypass re-entry using well established techniques and devices to traverse occlusions. These techniques could include mechanically disrupting the lesion using, e.g., the Silverhawk Plaque Excision System (available from FoxHollow Technologies, Redwood City, Calif.), or the OUTBACK® LTD® Re-Entry Catheter (available from Cordis Corporation). Alternatively, the techniques could involve gently navigating around the lesion in the subintimal space.

The location of the occlusion being known, it is possible to position the magnet at the catheter tip at a point just downstream of the occlusion, using imaging and/or image-guided methodologies used in the art. If required, the catheter tip may also be equipped with a tool for disrupting or penetrating the occlusion. In other embodiments, the occlusion site had been previously disrupted before inserting the intralumenal magnet-tipped catheter. The first magnet is described in further detail elsewhere herein, but it comprises a docking piece. As described further herein, a second magnet tipped catheter is navigated through the perivascular space to dock with the first magnet on the first catheter. In some embodiments, positioning of the second magnet is assisted by a light on the end of the first catheter, adjacent the first magnet.

At 212, three ports are introduced into the patient, for carrying out the deployment of the blood vessel. The three ports respectively accommodate a manipulation device, an imaging device, and the bypass blood vessel. The three ports are typically used in connection with, for example, a thoracoscopic surgical technique, or a laparoscopic surgical technique. The manipulation device is typically a catheter-based instrument or instruments that can perform various manipulations on tissue in the region of the occlusion, such as grabbing, tearing, pushing, pulling, or cutting. The imaging device is one typically used in surgical procedures of this type and may comprise a camera, an ultrasonic probe, or some other detector, and also typically includes a light source. The port that admits the bypass blood vessel is one that provides entry of a catheter to a region of the body close to a vein or artery from which blood flow will be diverted into the damaged artery downstream of the occlusion. The use of three ports is not a requirement to practice the technology herein. Fewer ports can be utilized using techniques well known in the art, such as single port laparoscopy that has been developed for several laparoscopic procedures.

At 214 a second catheter is introduced through the third of the just-described ports. This catheter is inserted so that its tip is positioned close to the location of the first magnet, at the tip of the first catheter, in the damaged artery. This can be accomplished by using the manipulation device to grab the tip of the second catheter, and with the assistance of the imaging device, drag the tip of the second catheter towards the required region. Alternatively, the second catheter may be made of a stiffer material and be steerable itself, with only minimal assistance from the manipulation device. In the case of introducing a bypass to a coronary artery, as further described herein, it is typically necessary to peel away a layer of the pericardium in the region of the occlusion in the damaged artery prior to subsequent steps. This is because the pericardium is thick enough to inhibit docking of the first catheter to a subsequently inserted second catheter. The peeling may be accomplished by cutting a partial perimeter of a region of the pericardium, and grabbing the region, pulling it out of the way, thereby opening a flap on the surface.

At 216 the second catheter can be withdrawn and reinserted, or replaced by another catheter, so that a catheter having a second magnet at its tip is inserted. The second magnet is thereby positioned close to the location of the first magnet in the damaged artery. The second magnet also comprises a docking piece, in particular one that is complementary to the docking piece on the first catheter.

At 218 the first and second magnets are docked to one another (sometimes termed mated where one magnet has a clearly discernible male portion and the other has a clearly discernible female portion) at the location in the damaged artery downstream of the occlusion. This constitutes a positive identification of the location of the tip of the first catheter, positioned inside the damaged artery, and ultimately enables the bypass blood vessel to be introduced and to accurately match up with the desired location on the damaged vessel. Docking can be achieved because the first and second magnets are strong enough and positioned close enough to one another to attract to one another; they are also preferably shaped so that they fit together firmly. Once docked, the first magnet and the second magnet are separated from one another only by the wall of the damaged artery. Successful docking can be facilitated if, for example, the first catheter is equipped with a light at its tip in addition to the first magnet so that the light is bright enough to be seen through the arterial wall. The light will be visible to the imaging device(s) deployed in the surgery and will thereby facilitate positioning of the tip of the second catheter close to the position of the first catheter tip.

Exemplary structures of the first and second magnets are further described elsewhere herein.

At 220 a perivascular guidewire is introduced down the lumen of the second catheter, and through a hole in the second magnet, to pierce the artery wall of the damaged artery at the location where the first and second magnets are attracted to one another. The docking piece on the first catheter is shaped to deflect and to force the guidewire into a downstream position within the damaged artery.

After the arterial wall has been pierced and the guidewire introduced into the lumen of the damaged vessel, the two magnets can be undocked. This can be accomplished either passively (by pulling back on both of the catheters), or actively. Examples of an active method of separating the magnets include a triggerable mechanical detachment such as a prong that can be advanced between the two magnets. In another embodiment, the magnetic force is created using electromagnets. In this case, the polarity of the magnets can be reversed, when desired, to drive the magnets apart. Alternatively, the magnetic forces can be switched off by turning off or reducing the electric current.

At 222 both the first and second catheters are removed from the patient.

At 224, an “introducer” is positioned over the guidewire and into the hole in the wall of the damaged artery downstream of the occlusion. The introducer is typically a plastic or rubber sheath that has a flexible and soft tapered tip that facilitates introduction of further components down the guidewire; it also enlarges the pierced hole in the damaged artery wall made by the guidewire.

At 226 the new blood vessel for effectuating the bypass, and having a stent and balloon or other anchoring device on its distal end, is run over the guidewire and through the introducer into the damaged artery. Aspects of the blood vessel are further described herein but typically may be several, or many tens of cm in length.

At 228 the introducer is removed.

At 230 the balloon at the distal end of the blood vessel is expanded to position the stent inside the damaged artery distal to the occlusion. This anchors the stent at that location. The stent typically is not straight but is kinked, for example by an angle between 60 and 90° to reinforce the junction between the bypass blood vessel and the damaged artery.

At 232 the proximal end of the blood vessel is joined at a position in a main artery, for example, a subclavian artery, proximal to the occlusion. This may be accomplished by surgical methods well-practiced in the art and may include making a small incision in the patient to reveal the situs. The proximal junction may be sewn by hand and may or may not require a second stent. It is typical that the main artery that is chosen has been clamped during the prior steps of the procedure, to minimize blood flow into the region of the bypass. Those clamps can now be removed.

In other embodiments, the proximal end of the blood vessel can be anchored using a system similar to that described for the distal end (i.e., an expandable stent or other anchoring device). During deployment of this stent, blood loss from the proximal source artery can be controlled using one or more inflatable balloons to exclude blood flow. These balloons can be shaped to have a lumen or other channel that allows blood flow through the source artery and to distal branches, but isolate the area of the artery that will be perforated to facilitate the bypass.

It is consistent with the methods herein that the anchoring of the proximal end of the bypass blood vessel can occur before the vessel is attached at the distal location.

The end result of the surgery is a bypass blood vessel that diverts a portion of blood flow from a proximal, main artery, into a distal location of a diseased vessel downstream of an occlusion.

### Catheters and Magnets

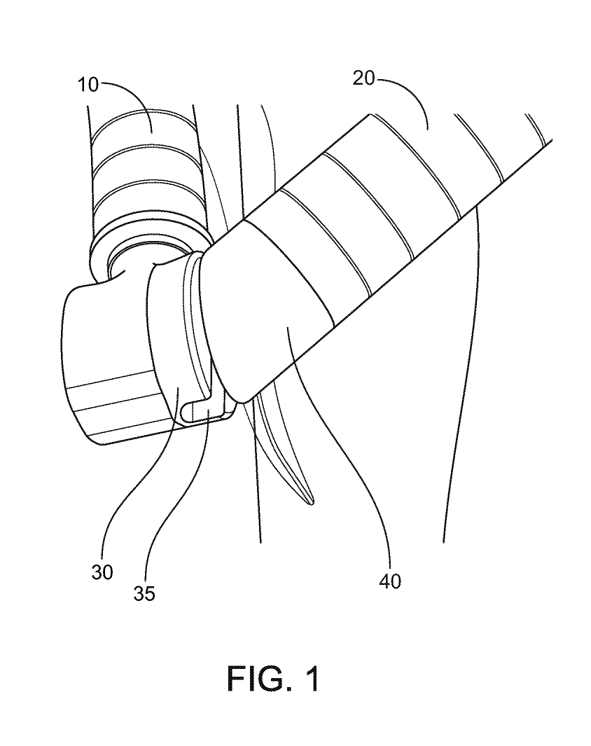

The procedure described herein is now illustrated in part by FIGS. 1-5. FIGS. 1-5 illustrate a coronary bypass but it would be understood that the general principles depicted are applicable to other percutaneous bypass procedures. Additionally, it would be understood that, although the procedures and devices herein utilize magnets for docking two catheters, other methods and devices for achieving that docking can be envisaged.

FIGS. 1-3 show a sequence of depictions of two catheters docking In FIG. 1, first catheter 10, positioned inside a damaged artery, has a first magnet 30 at its tip. Second catheter 20, positioned outside the damaged artery, has a second magnet 40 at its tip. First magnet 30 and second magnet 40 are shown docked to one another, separated only by the wall of the damaged artery. As shown, first magnet 30 occupies a portion of the tip of first catheter 10, and second magnet 40 occupies the entirety of the tip of second catheter 20. In other embodiments, not shown, the entirety of the respective tip(s) of both catheters is magnetic.

A surface of first magnet 20 is complementary to a surface of second magnet 40. The complementarity shown in FIGS. 1-3 is illustrative and not limiting. In particular, the shape of the first and second magnets in FIGS. 1-3 is such that a face of the first magnet is disposed outwardly from the axis of the first catheter, and a face of the second magnet is disposed at an angle between 0 and 90° to the axis of the second catheter. This arrangement of the respective faces permits the second catheter to dock to the first at an acute angle. In other embodiments, not shown, the first and second magnets have geometric features that cause them to snap together with a fixed alignment. In other embodiments the first and second magnets have geometrically complementary shapes, in the manner of a mechanical key that permit them to dock together in a single orientation and, while docked together, experience reduced degrees of freedom of movement relative to one another.

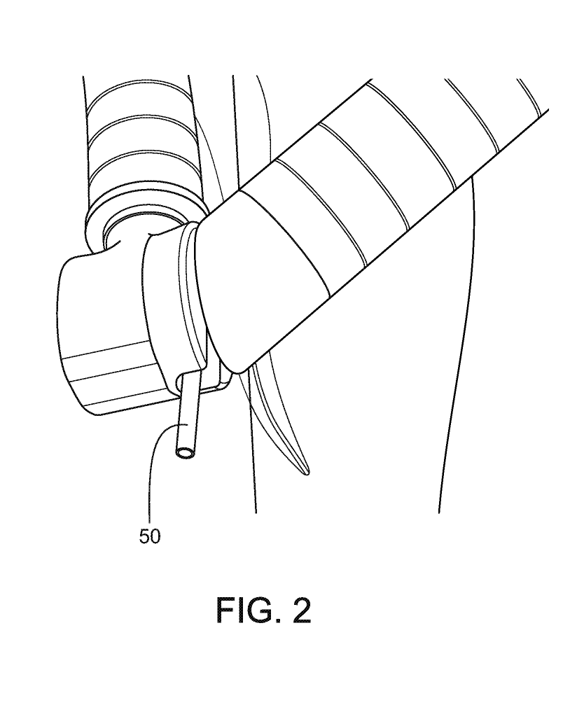

FIG. 2 illustrates a feature of the second magnet, which is that it has a concentric hole to permit a guidewire to travel down through it. Such a configuration of second magnet 40 is not limited to a concentrically disposed hole; if a hole is present it can be located at an off-center or off-centroid location. It is also possible to use a magnet having a cutout at one edge, i.e., not a cutout that is fully enclosed, that guides a guidewire.

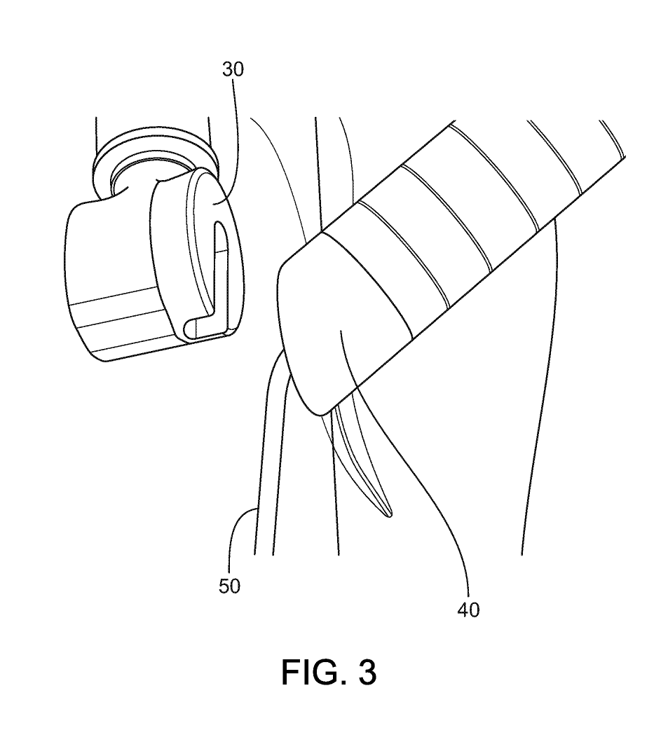

One important feature of the magnet 30 is that it is shaped to allow the guidewire from the proximal end to pierce through the vessel wall without hindrance from the interior side of the artery, and then to deflect down into the distal target vessel such that the second catheter in the distal vessel can be withdrawn. The first magnet 30 can be, for example, clam shelled or scalloped to allow this redirection. FIG. 2 illustrates this feature of the first magnet, which is that the surface that is complementary to the second magnet contains a recessed portion, such as a chute, groove or a slot 35, for accommodating and directing a guidewire. The method as performed and as further described herein utilizes a guidewire which is introduced via the second catheter into the region where the two magnets are docked to one another. The guidewire must not terminate at the surface of the first magnet but must be caused to emerge from the junction between the first and second magnets. The groove or slot is typically wider than the diameter of the guidewire and may be curved in cross-section to facilitate deflecting the tip of the guidewire when it is inserted. The groove or slot is also typically smooth on its surface so as to provide minimal resistance (via friction) or obstacles to motion of the tip of the guidewire. The groove need not traverse the entire surface of the first magnet but, as shown in FIG. 3, just occupy a fraction of the width.

FIG. 3 shows the first magnet being withdrawn from the region where the first and second magnets were docked. Methods of achieving an uncoupling of the two magnets have been described elsewhere herein. Also shown in FIG. 3 is the guidewire, having been inserted further, and now traveling inside the damaged artery, distal to and downstream from, the occlusion.

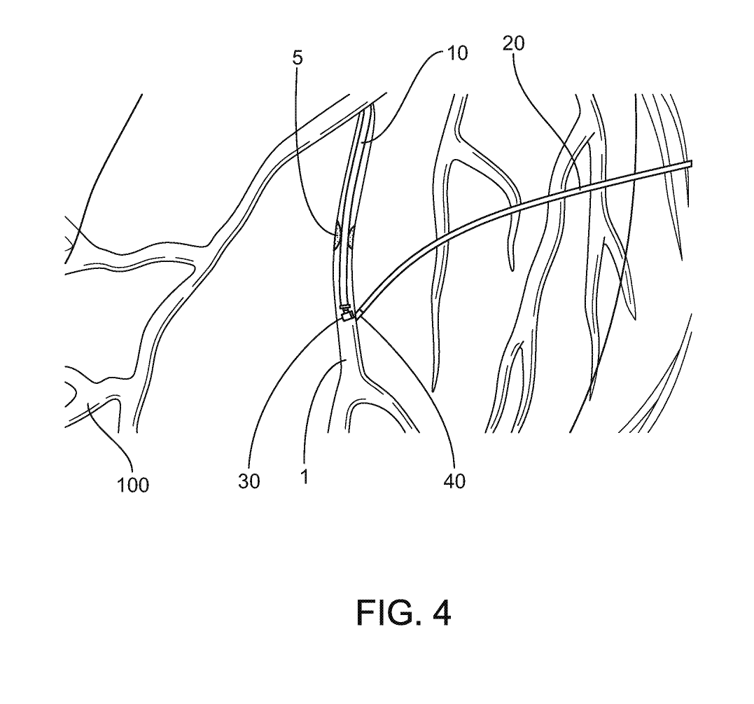

FIG. 4 shows a view of the damaged artery 1, with occlusion 5, and two catheters 10 and 20 docking to one another via magnets 30 and 40 at a point in the damaged artery downstream of the occlusion. A sampling of the remainder of the patient's vasculature 100 is also visible.

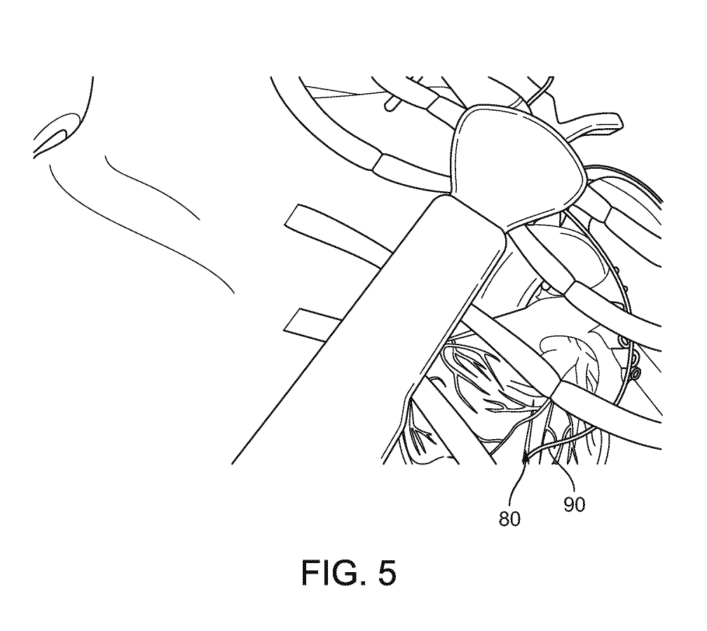

FIG. 5 shows a cutaway view of a patient's chest showing a bypass vessel 90 in place, attached to a site 80 of a diseased vessel.

Bypass vessel 90 can be made from many suitable materials. Of particular use are tissue-engineered sheets, as described in U.S. Pat. Nos. 6,503,273, 7,112,218, 7,166,464, and 7,504,258, and U.S. Patent Application Publication No. 2010-0040663 (“Arterial Implants”), all of which are incorporated herein by reference in their entirety.

### EXAMPLES

### Example 1

### Coronary Percutaneous Bypass

According to the technology herein, percutaneous delivery of a bypass conduit, to a distal coronary artery, is performed by deploying two catheter-based systems. A first catheter system is advanced into the coronary artery to a target location distal to the blockage. The second catheter system is advanced into the aortic arch, typically from either a femoral artery or an upper trunk artery such as the subclavian, to a position proximal to the occlusion. It is a goal of the procedure to connect the target location distal to the occlusion, via use of the second catheter, to a proximal source of blood supply outside of the heart. As used herein, delivery of a device or the bypass vessel includes positioning the device or vessel at the desired location. Deployment of the device comprises securing it in position, e.g., by inflating a balloon on the interior of a stent.

The wires of both the first and second catheter systems serve as guidewires for subsequent steps of the method described herein.

Once both guide catheters have been placed, in one embodiment the bypass vessel is delivered and deployed at the distal location, and subsequently deployed at the proximal location. The bypass vessel can be attached to the distal location via a device.

In certain embodiments, the proximal anchor point can be in the aorta instead of in, e.g., a narrower artery such as the subclavian artery. In such embodiments, it would be necessary to perforate the aorta. This might be a preferred approach in certain patients if higher blood flow is needed, or where the narrower arteries cannot support such bloodflow. In such applications, for example, the proximal anchoring device can be a bifurcating stent graft that has a large diameter proximal neck that is deployed in the aorta and a small diameter side branch that will perforate the aorta and direct the bypass vessel, when positioned, to target the distal location of the damaged coronary vessel. The bifurcated stent graft can be comprised of a resorbable or permanent stent. The stents can be either balloon or self-expanding. The membrane surrounding the stent can be either synthetic (ePTFE, Dacron, PE or other materials) or biological (sheet-based tissue engineered fibroblasts, peritoneum, pericardium, dermis, small intestine sub mucosa, native vein or artery etc.) in nature. The device can include branching systems based on either a fenestration approach or a chimney approach. The device can also include a pre-manufactured branch.

Additional difficulties of using the aorta as a proximal site arise because blood flow needs to be restricted during the procedure, and it is not feasible to cross-clamp the aorta for a long time (such as an hour or more). Accordingly, additional strategies can be deployed, such as, but not limited to one or more balloons configured to exclude blood flow from the working area and having a lumen in between them. For example, a pair of balloons situated upstream and downstream respectively and separated by a tube so that a reduced blood flow is directed down the tube but the work-site remains clear. A similar structure to permit the catheter to be inserted may also be utilized.

To deploy the stent-graft in the aorta, first the upstream stent is deployed, taking care not to exclude other branches on the aorta. If upstream branches must be covered, flow can be restored using other techniques known in the art, with either fenestrating or chimney style side branches. Either the original guidewire or a second cutting device is then inserted into the aorta following the original guidewire. This cutting device must be inserted into the lumen of the short side branch of the stent device, or must pierce through the wall of the stent device. This cutting device can have a lumen or a multiplicity of lumens such that other devices may be advanced through the side branch. Cutting can be achieved by tearing, cutting, or burning through the wall of the stent-graft and/or the aorta. This list is not meant to be limiting: other ways of piercing through the stent-graft and aorta walls can be envisioned. Moreover, the cutting catheter can have a balloon which can be inflated to limit blood flow out through the side branch after cutting and during further manipulations. Once the aorta (and if necessary the stent graft) is cut, both the side branch and/or the guidewire can be advanced into the perivascular space.

In order to make a connection with the distal target location of the damaged coronary artery, a guidewire is advanced through the side branch of the aorta stent graft. This guidewire can be advanced via the previous catheter, or can be a separate device. This guidewire, sometimes called a bridge, can be a steerable catheter equipped with a means of direct visualization to help locate the distal target location. A video or still camera, or fiber optic cable are preferred visualization equipment. Angiographic imaging can also be used to help guide a radio-opaque guidewire system across the perivascular space. The bridge catheter can also be equipped with a means of actively engaging the catheter tip at the distal target location of the coronary artery. This engagement can be facilitated by a mechanical capture device such as a loop, or can include a directed capture device such as a magnet or electromagnet as described elsewhere herein.

In a preferred embodiment, the bridge catheter is steered toward the distal coronary target by using a combination of angiography and direct visualization using a fiber optic cable that can be advanced toward a light source (which is mounted to the coronary target guidewire) in the coronary artery.

In order to advance to within a few millimeters of the coronary target, it is preferable to pierce the pericardium and drain the fluid. This can be done with a cutting tip on the bridge catheter or via a separate catheter delivered through the lumen of either guidewire. The cut can be originated from either the inside or the outside, but in one embodiment, the steerable catheter would perform all navigation and cutting actions. The bridge catheter is advanced through the hole in the pericardium and toward the distal target location of the damaged coronary artery. The same cutting device can be used to cut the coronary artery to accommodate the bypass blood vessel. In order to make targeting more accurate, the bridge wire is actively and positively coupled to the coronary artery catheter. This can be achieved via magnetic attraction between the tips of the two wires, or via a mechanical coupling such as a loop or a key/hole. Such couplings serve first to positively engage the two catheter tips at a desired location on the vessel, and second to facilitate piercing of the vessel wall at that location. Thereafter, the bypass vessel can be coupled between the proximal location in the aorta and the distal location on the coronary artery, for example, by using the bridge catheter and/or a guidewire. When the proximal end is in the aorta, it is typically deployed first (before the distal connection), by inflating a balloon inside the anchoring stent, because the aorta is so large and the blood flow there is significantly higher than at the distal location.

In another approach to a percutaneous coronary bypass, the proximal end of the percutaneous bypass originates in one of the internal mammary arteries, instead of the aorta. In this embodiment, a cutting balloon with a blade oriented to make a transverse cut is advanced into the mammary artery. The cutting balloon comprises a combination of a catheter with balloon at the end to apply pressure and a blade to cut, e.g., a vessel. Proximal to the cut, a balloon with a lumen or multiplicity of lumens is expanded to exclude blood flow (or at least limit blood loss out of the mammary). Distal to the location where the mammary artery is to be cut, it must be closed by ligation, embolization, or cauterization etc. to limit retrograde bleeding. The cutting balloon is then activated and a transverse cut made in the artery.

Other approaches to cutting an arterial wall from the interior are consistent with the methods described herein. For example, in place of a cutting balloon, a combination of, separately, a cutting blade and a balloon to stench blood flow could achieve the atherectomy. Other configurations of cutting balloon known in the art can also be suitably adapted or configured to perform the atherectomy. For example, a balloon with a triggerable blade, controllable remotely, could be utilized; such a balloon could perform multiple roles, of excluding blood flow, inflating a device, as well as cutting the arterial wall.

The balloon used to exclude blood from the mammary artery is momentarily deflated, and the bypass stent graft is advanced past the balloon such that the device sits entirely distal to the balloon. The stent graft will likely extend out past the end of the mammary artery and into the perivascular space at this point. The proximal end of the stent graft is then deployed to secure the device in the mammary artery. In order to join the distal end of the stent graft to the coronary artery target (distal to the coronary artery occlusion), a guidance system is used as described hereinabove. Once the guidewire has bridged the gap to the coronary artery, the distal end of the stent graft is deployed. Blood flow is restored by deflating the balloon and withdrawing both sets of catheters.

In another similar example, the bypass can originate from another proximal artery, such as the subclavian artery or the humeral artery. Use of such a proximal target can further simplify the proximal anastomosis, as these targets can be accessed with minimally invasive or open techniques with few complications. The bypass conduit is then navigated through the perivascular space to couple with the first catheter to make the distal anastomosis as described above.

### Example 2

### Peripheral Renal Bypass

A similar approach to that of Example 1 can be used for peripheral bypass or other peripheral rerouting of blood flow, to create AV shunts for example. In order to restore blood flow to a diseased or damaged portion of a patient's kidney, for example an open anastomosis can be made at the iliac artery for the proximal connection, and then the conduit can be delivered through the perivascular space to the renal artery using the guiding techniques described herein. This type of approach is appropriate and useful for debranching procedures where the renal arteries have been occluded.

The foregoing description is intended to illustrate various aspects of the instant technology. It is not intended that the examples presented herein limit the scope of the appended claims. The invention now being fully described, it will be apparent to one of ordinary skill in the art that many changes and modifications can be made thereto without departing from the spirit or scope of the appended claims.

What is claimed:

1. An apparatus for coupling two catheters across a blood vessel wall, the apparatus comprising:
a first catheter having a first magnet located at its end,
a second catheter having a second magnet located at its end;
wherein a first surface of the first magnet is complementary to a second surface of the second magnet;
wherein the first magnet is strong enough to attract and engage the second magnet when separated from the second magnet by the blood vessel wall, and
wherein the second magnet encloses a hole through which a guidewire travels; and
wherein the first magnet comprises a chute that accepts the guidewire configured to deflect a guidewire delivered through the second magnet downstream into the blood vessel.

2. A method of using the apparatus of claim 1 to carry out a percutaneous bypass.

3. The method of claim 2, wherein the bypass is a percutaneous coronary bypass.

4. A method of performing a percutaneous bypass on a subject, the method comprising:
docking a first catheter situated inside a damaged vessel to a second catheter situated outside the vessel, at a location downstream of an occlusion in the damaged blood vessel; and
inserting a bypass blood vessel over the second catheter.

5. The method of claim 4, wherein the first and second catheters have magnetic tips that are complementary to one another.

6. A method of performing a percutaneous bypass on a subject, the method comprising:
introducing a first catheter into the subject;
positioning a first tip of the first catheter at a location distal to an occlusion in a damaged blood vessel;
introducing a second catheter into the subject at a location proximal to the occlusion;
docking a second tip of the second catheter to the first tip of the first catheter at the location distal to the occlusion;
inserting a guidewire down the second catheter so that the guidewire traverses the location distal to the occlusion;
withdrawing the second catheter;
inserting a length of bypass blood vessel over the guidewire; and
joining the bypass blood vessel to the damaged blood vessel at the distal location.

7. The method of claim 6, wherein the first tip comprises a first magnet, and the second tip comprises a second magnet and the docking comprises a magnetic attraction.

8. The method of claim 6, wherein, prior to withdrawing the second catheter, the first and second tips are undocked from one another.

9. The method of claim 6, further comprising carrying out an anastomosis at a proximal location.
